(19) United States
(12) Patent Application Publication
(10) Pub. No.: US 2025/0303015 A1
(43) Pub. Date: Oct. 2, 2025

(54) Fragrance Distribution Drone and Distribution System

(71) Applicant: ELC MANAGEMENT LLC., Melville, NY (US)

(72) Inventor: Christopher Aidan, Austin, TX (US)

(21) Appl. No.: 18/622,762

(22) Filed: Mar. 29, 2024

(51) Int. Cl. A61L 9/14; B64U 20/80; B64U 101/45

(52) U.S. Cl. CPC A61L 9/14; B64U 20/80; A61L 2209/111; A61L 2209/134; B64U 2101/45

(57) ABSTRACT

A drone-based fragrance distribution system includes a fragrance distribution drone and a user input system. The fragrance distribution drone includes a drone body, a navigation system connected to the drone body, a fragrance housing connected to the drone body, a fragrance dispersal system connected to the drone body, a drone wireless communication system connected to the drone body, and a drone controller in communication with the navigation system, the fragrance dispersal system, and the wireless communication system. The user input system includes a control wireless communication system selectively in communication with the drone wireless communication system, a user interface, and an input controller in communication with the control wireless communication system.

### FIELD OF THE DISCLOSURE

The present disclosure generally relates to fragrance distribution devices and systems. More specifically, the present disclosure relates to a fragrance distribution drone and a drone-based fragrance distribution system.

### BACKGROUND

In both residential and commercial environments, fragrances are often distributed to enhance an environment with a particular scent and/or cover unpleasant odors. Common fragrance distribution devices include candles, air fresheners, and oil diffusers. Some HVAC systems can be used to distribute a fragrance. Disadvantageously, these devices and systems typically are limited by static placement, provide uneven distribution of the fragrance, and lack active control over their operation.

### SUMMARY

Examples within the scope of the present disclosure are directed to approaches for distributing a fragrance using a fragrance distribution drone.

In an example, a fragrance distribution drone includes a drone body, a navigation system connected to the drone body, a fragrance housing connected to the drone body, a fragrance dispersal system connected to the drone body, and a controller in communication with the navigation system and the fragrance dispersal system, the controller including a non-transitory memory and one or more processors. The non-transitory memory stores instructions that, when executed by the one or more processors, cause the controller to control the fragrance dispersal system and the navigation system to at least one of: distribute a fragrance at a predetermined time while executing a flight pattern, distribute a fragrance at a target location while executing a flight pattern, achieve a target fragrance concentration at a target location while executing a flight pattern, distribute a fragrance while executing a flight pattern in response to an activating event, distribute a plurality of fragrances simultaneously at a target ratio while executing a flight pattern, and distribute a plurality of fragrances sequentially while executing a flight pattern.

In an approach, at least one sensor may be configured to collect sensor data and provide the sensor data to the controller, the sensor data indicating a change in a quantity of fragrance within the fragrance housing. The instructions, when executed by the one or more processors, may further cause the controller to adjust operating parameters of the navigation system in response to the sensor data indicating the change in the quantity of fragrance within the fragrance housing.

In an approach, the operating parameters may include at least one of a propeller speed, quantity of propellers of a plurality of propellers in operation, and location of propellers of a plurality of propellers in operation.

In an approach, at least one sensor may be configured to collect sensor data and provide the sensor data to the controller. The sensor data may include at least one of: a location of the drone, a type of detected fragrance, a fragrance concentration, a proximity to a person or object, an identification of a person or object, a rate of environmental airflow, and a direction of environmental airflow.

In an approach, in response to the sensor data, the instructions, when executed by the one or more processors, may further cause the controller to control at least one of the fragrance dispersal system and the navigation system to at least one of: distribute a fragrance, adjust a rate of distribution of a fragrance, cease distribution of a fragrance, adjust a rate of distribution or cease distribution of at least one of the plurality of fragrances set to achieve the target ratio, adjust the target ratio, adjust the predetermined time at which a fragrance is to be distributed while executing a flight pattern, adjust the target location, trigger a flight pattern of the navigation system, adjust the flight pattern of the navigation system, and end the flight pattern of the navigation system.

In an approach, the fragrance housing may be configured to selectively secure and release a fragrance cartridge.

In an approach, wherein the instructions, when executed by the one or more processors, may further cause the controller to control the fragrance dispersal system and the navigation system according to one of a plurality of modes, the plurality of modes including an intensive mode for rapid fragrance distribution and a maintenance mode for sustaining a consistent fragrance concentration.

In an example, a drone-based fragrance distribution system includes a fragrance distribution drone having a drone body, a navigation system connected to the drone body, a fragrance housing connected to the drone body, a fragrance dispersal system connected to the drone body, a drone wireless communication system connected to the drone body, and a drone controller in communication with the navigation system, the fragrance dispersal system, and the wireless communication system. The drone controller includes a non-transitory memory and one or more processors. The drone-based fragrance distribution system further includes a user input system having a control wireless communication system selectively in communication with the drone wireless communication system, a user interface, and an input controller in communication with the control wireless communication system. The input controller includes a non-transitory memory and one or more processors.

In an approach, the user interface may be configured to receive user input including a selection of at least one of: a fragrance type, a target fragrance concentration, a target ratio of a plurality of fragrances, a sequential order for distribution of a plurality of fragrances, a predetermined time for fragrance distribution, a target location for fragrance distribution, and an activating event for fragrance distribution.

In an approach the non-transitory memory of the drone controller or the non-transitory memory of the input controller may store instructions that, when executed by the one or more processors of the drone controller, cause the drone controller, in response to the input controller activating the user interface, to control at least one of the fragrance dispersal system and the navigation system to at least one of: distribute a fragrance, adjust a rate of distribution of a fragrance, cease distribution of a fragrance, adjust a rate of distribution or cease distribution of at least one of the plurality of fragrances set to achieve the target ratio, adjust the target ratio, adjust the predetermined time at which a fragrance is to be distributed while executing a flight pattern, adjust the target location, trigger a flight pattern of the navigation system, adjust the flight pattern of the navigation system, and end the flight pattern of the navigation system.

In an approach, the input controller may be configured to store a selection at the user interface to a user profile, and the non-transitory memory of the input controller may store instructions that, when executed by the one or more processors of the input controller, cause the input controller to activate the user interface when triggered by an activating event.

In an approach, the input controller may be configured to store a plurality of user profiles.

In an approach, an activating event may be user input at the user interface or electronic recognition of a person or object by the user input system or by the fragrance distribution drone.

In an approach, wherein the non-transitory memory of the drone controller or the non-transitory memory of the input controller may store instructions that, when executed by the one or more processors of the drone controller, cause the drone controller, in response to the input controller activating the user interface, to control at least one of the fragrance dispersal system and the navigation system to at least one of: distribute a fragrance, adjust a rate of distribution of a fragrance, cease distribution of a fragrance, adjust a rate of distribution or cease distribution of at least one of the plurality of fragrances set to achieve the target ratio, adjust the target ratio, adjust the predetermined time at which a fragrance is to be distributed while executing a flight pattern, adjust the target location, trigger a flight pattern of the navigation system, adjust the flight pattern of the navigation system, and end the flight pattern of the navigation system.

In an approach, the drone-based fragrance distribution system may further include a docking device configured to connect to the fragrance distribution drone, a rechargeable battery may be connected to the drone body, and the docking device may be configured to electrically charge the rechargeable battery.

In an approach, the fragrance dispersal system may include a plunger to increase pressure within the fragrance housing and a tube to siphon a fragrance from the fragrance housing.

In an approach, the navigation system may include a propeller operably connected to a motor, and the motor of the navigation system may also be operably connected to the plunger of the fragrance dispersal system.

In an approach, the non-transitory memory of the drone controller may store instructions that, when executed by the one or more processors of the drone controller, cause the drone controller to provide drone information and send the drone information via the drone wireless communication system to the control wireless communication system. The non-transitory memory of the input controller may store instructions that, when executed by the one or more processors of the input controller cause the input controller to display the drone information on the user interface or send the drone information via the control wireless communication system.

In an approach, the drone information may be at least one of: a battery level, fragrance quantity onboard the fragrance distribution drone, maintenance reminder, fragrance usage patterns, and drone operation times.

In an approach, the drone-based fragrance distribution system may include a plurality of fragrance distribution drones.

In an example, a drone-based method of fragrance distribution includes providing a fragrance distribution drone including a fragrance dispersal system and further including at least one sensor. The method further includes flying the fragrance distribution drone within an environment on a baseline flight plan. The method further includes collecting sensor data using the at least one sensor of the fragrance distribution drone during the baseline flight plan, and analyzing, using a processor, the sensor data. The method further includes optimizing, using a processor, a subsequent flight plan and operation of the fragrance dispersal system based on the sensor data to achieve at a target fragrance concentration within the environment while avoiding obstacles within the environment. The method further includes operating the fragrance distribution drone based on the optimized subsequent flight plan and the optimized operation of the fragrance dispersal system to achieve the target fragrance concentration within the environment while avoiding the obstacles within the environment.

In an approach, the drone-based method of fragrance distribution may further include flying a plurality of fragrance distribution drones simultaneously on coordinated baseline flight plans, collecting the sensor data using at least one sensor of each of the plurality of fragrance distribution drones, and optimizing, using a processor, the subsequent flight plan of each of the plurality of fragrance distribution drones based on the sensor data to achieve at a target fragrance concentration within the environment while avoiding obstacles within the environment.

### BRIEF DESCRIPTION OF THE DRAWINGS

The above needs are at least partially met through provision of one, more than one, or any combination of the approaches described in the following detailed description, particularly when studied in conjunction with the drawings, wherein:

FIG. 1 illustrates schematically a fragrance distribution drone in accordance with various examples.

FIG. 2 illustrates a fragrance dispersal system and fragrance housing of the fragrance distribution drone of FIG. 1 in accordance with various examples.

FIG. 3 illustrates schematically the controller of the fragrance distribution drone of FIG. 1 in accordance with various examples.

FIG. 4 illustrates schematically a drone-based fragrance distribution system in accordance with various examples.

FIG. 5 illustrates schematically the input controller of the user input system of the drone-based fragrance distribution system of FIG. 4 in accordance with various examples.

FIG. 6 illustrates schematically a docking device configured to connect to a fragrance distribution drone in accordance with various examples.

FIG. 7 illustrates schematically a drone-based method of fragrance distribution.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted in order to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various approaches, a fragrance distribution drone is provided as part of a drone-based fragrance distribution system that enables targeted delivery of fragrances in a customizable and mobile manner. Because the fragrance distribution drone is cable to fly to different locations within an environment (either residential or commercial), the dispersal of a fragrance is not limited to one or even a plurality of static locations. Instead, the fragrance can be delivered to virtually any location within the environment. Further, the fragrance distribution drone can include sensors to assess the efficacy of fragrance delivery and make adjustments to either a navigation pattern or a delivery technique as a result. In combination with a user input system, the fragrance delivery can be customized for any number of parameters, including frequency of delivery, location of delivery, type of fragrance, and more. In this way, the fragrance distribution system provides more effective and customizable fragrance delivery than known fragrance distribution devices.

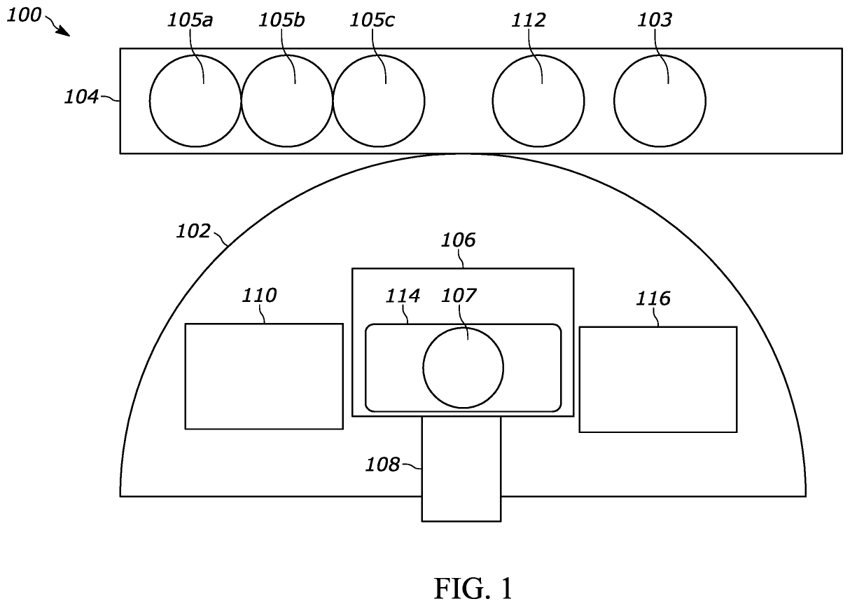

FIG. 1 illustrates schematically a fragrance distribution drone 100. As shown, the fragrance distribution drone 100 includes a drone body 102. The drone body 102 is a frame or chassis that serves as the structural foundation for the fragrance distribution drone 100. A navigation system 104 is connected to the drone body 102. The navigation system 104 includes the structural components necessary for the fragrance distribution drone 100 to move through the air, such as a motor 103 and a propeller 105. In some arrangements, the navigation system may include a plurality of propellers 105 (e.g., 105a, 105b, 105c), which may be symmetrically distributed relative to a center of the drone 100. The navigation system 104 further includes components necessary to guide movement of the fragrance distribution drone 100, such as a global positioning system (GPS), inertial measurement unit (IMU) that may include including accelerometers and gyroscopes to measure acceleration and angular rate, barometer and altitude sensors to measure changes in altitude, a compass to provide orientation information, and a vision system including a camera for obstacle detection.

A fragrance housing 106 is connected to the drone body 102. In the arrangement shown in FIG. 1, the fragrance housing 106 is positioned toward a center of the drone body 102. The fragrance housing 106 is configured to hold a fragrance 107, which may be a solid or a liquid. In FIG. 1, the fragrance housing 106 is configured to selectively secure and release a fragrance cartridge 114. The fragrance cartridge 114 is a container or device designed to hold and selectively release the fragrance 107. In other arrangements, the fragrance 107 may be directly placed within the fragrance housing 106. A fragrance dispersal system 108 enables distribution of the fragrance from the fragrance housing 106. The fragrance dispersal system 108 may actively expel the fragrance from the fragrance housing 106 by, for example, spraying liquid fragrance 107 into the air. Alternately, the fragrance dispersal system 108 may passively allow fragrance distribution by, for example, simply opening a door to allow air flow to and from the fragrance housing 106. The fragrance dispersal system 108 may include any mechanical or electronic features necessary to distribute the fragrance 107, such as a pump, motor, circuitry, passageway, or fan. In some arrangements, the fragrance distribution drone 100 may include a plurality of fragrance housings 106, each configured to contain a different fragrance 107, and the fragrance dispersal system 108 may be configured to selectively disperse each fragrance 107 alone or in combination.

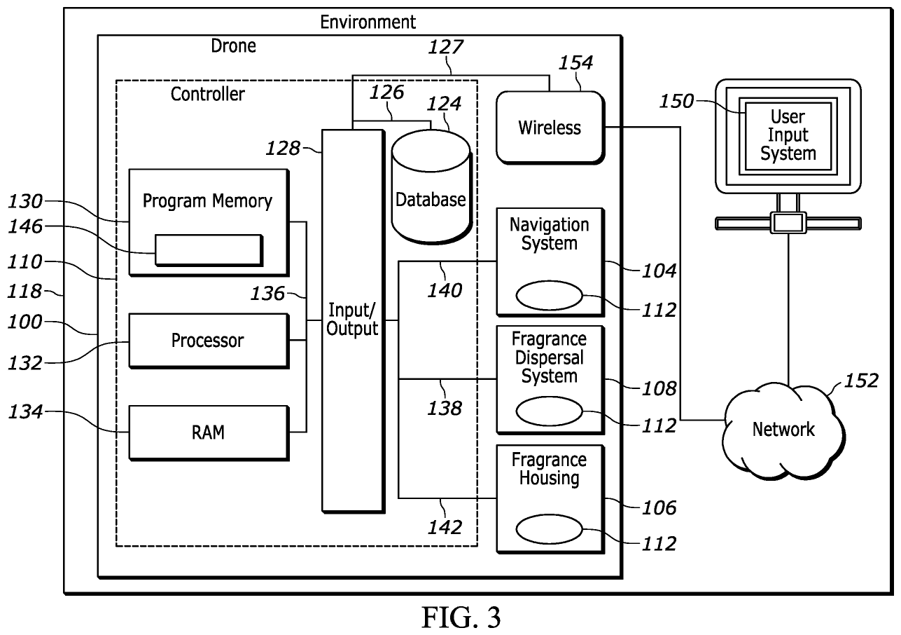

A controller 110 is in communication with the navigation system 104 and the fragrance dispersal system 108. The controller 110 is described in greater detail with respect to FIG. 3. One of the functions of the controller 110 is to receive sensor data. The fragrance distribution drone 100 includes at least one sensor 112 in communication with the controller 110. The sensor 112 may be part of the navigation system 104 (e.g., the accelerometers, gyroscopes, barometer, and altitude sensors discussed above). The sensor 112 may be located at or within the fragrance housing 106 (e.g., as a pressure sensor detecting an amount of fragrance 107 contained within the fragrance housing 106) or at or within the fragrance dispersal system 108 (e.g., as a flow sensor monitoring the rate at which fragrance 107 is expelled). Typically, the fragrance distribution drone 100 will include a plurality of sensors 112 of many different types. Sensor data collected from the sensors 112 may include a location of the drone 100, a type of detected fragrance 107, a fragrance concentration, a proximity to a person or object, an identification of a person or object, a rate of environmental airflow, and a direction of environmental airflow.

A battery 116 is connected to the drone body 102 and in electrical communication with the drone body 102, the navigation system 104, the fragrance housing 106, the fragrance dispersal system 108, and/or the controller 110. The battery 116 may be rechargeable, as discussed in further detail below.

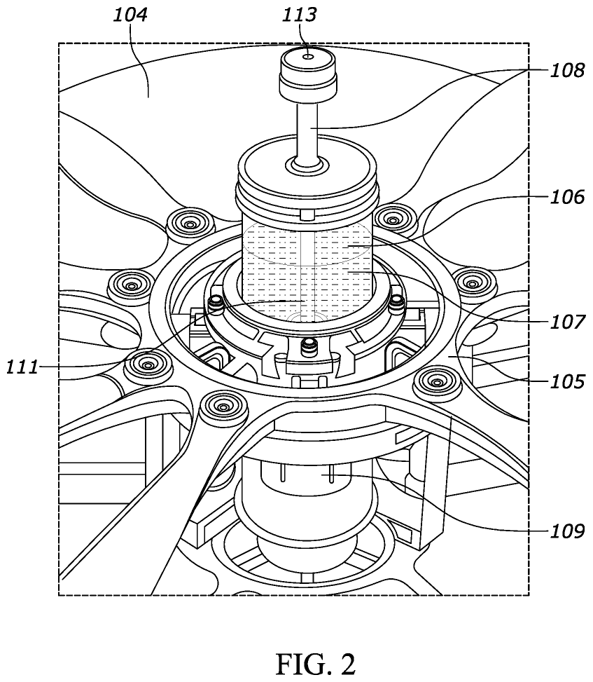

FIG. 2 illustrates a fragrance dispersal system 108 and fragrance housing 106 of the fragrance distribution drone 100 of FIG. 1 in accordance with various examples. In the arrangement shown in FIG. 2, the fragrance housing 106 is a cylindrical container holding liquid fragrance 107. The navigation system 104 includes a propeller 105, and the propeller 105 is connected to a plunger 109 directly or indirectly. For example, in the arrangement shown, housing 106 has an open bottom in which the plunger 109 is sealingly positioned to define the cavity containing the fragrance 107. The plunger 109 is threadedly connected to the propeller 105 such that rotation of the propeller 105 results in linear movement of the plunger 109. In some arrangements, a cam may be provided between the propeller 105 and the plunger 109 such that the linear movement of the plunger 109 occurs only when the cam (e.g., a tooth on the threaded connection) engages the plunger 109 to ensure that the respective rates of rotary movement of the plunger 109 and linear movement of the plunger 109 are linked in an optimal manner for both navigation and fragrance distribution. Linear movement of the plunger 109 increases pressure within the fragrance housing 106, which is sealed to be airtight but for a tube 111 connecting the interior of the fragrance housing 106 to the environment. In the arrangement shown, the tube 111 has an egress aperture 113 located above the fragrance housing 106. As pressure increases within the fragrance housing 106 to be greater than the ambient pressure, fragrance 107 within the fragrance housing 106 is siphoned into the environment through the egress aperture 113. In arrangements of the fragrance distribution drone 100 containing multiple propellers 105 (e.g., 105a, 105b, and 105c), a separate fragrance housing 106 may be provided for each propeller 105 and a unique fragrance 107 may be contained in each fragrance housing 106.

In other arrangements, the fragrance dispersal system 108 may not include a plunger 109. Instead, motion of the propellers 105 may create a pressure differential above the tube 111 that naturally draws the fragrance 107 from the fragrance housing 106 without the need for the additional pressure provided by a moving plunger 109. A nozzle or other distribution device may be provided on the tube 111, and the nozzle and/or tube 111 may include capillaries to use capillary action to draw the fragrance 107 out for dispersal. Other options for the fragrance dispersal system 108 include a centrifugal force system whereby rotation of the propeller 105 exerts a centrifugal force on the fragrance 107 to push it through the tube 111. In some arrangements, the fragrance dispersal system 108 may include a pump.

In another variation of the fragrance dispersal system 108 and fragrance housing 106, the fragrance housing 106 may be a connection on the drone body 102, and the fragrance dispersal system 108 may be a material infused with the fragrance 107 and secured to the drone body 102. The distribution of the fragrance 107 occurs via airflow, generated in part by the propeller 105 or other components of the navigation system 104.

Other variations of the fragrance dispersal system 108 and fragrance housing 106 are also within the scope of the present disclosure. For example, the plunger 109 shown in FIG. 2 may be configured to actively push the fragrance 107 out of an aperture located in a lower portion of the fragrance housing 106 rather than to create a siphon. As another example, the motor 103 of the navigation system 104 may be configured to impart rotary motion to both the propeller 105 and to an impeller located within a fragrance housing 106. The fragrance housing 106 may include a centrifugal pump casing in which the impeller is located, and the centrifugal pump housing may define a fluid flow path for the fragrance 107 from the fragrance housing 106 to the ambient environment. Operation of the motor 103, which causes the impeller to rotate within the centrifugal pump housing, may cause the fragrance 107 to be pumped along the fluid flow path and dispersed in the environment.

FIG. 3 is an example block diagram of an example controller 110, illustrating the various components used in implementing an example embodiment of a fragrance distribution drone 100. The controller 110 is configured to execute the functions of the disclosed embodiments in order to distribute a fragrance 107 in an environment 118. The controller 110 may be operatively connected to a database 124 via a link 126 connected to an input/output (I/O) circuit 128. The database 124 may, for example, store sensor data collected from any sensor 112. It should be noted that, while not shown, additional databases may be linked to the controller 110 in a known manner. The controller 110 includes a program memory 130, one or more processors 132 (may be called microcontrollers or a microprocessors), a random-access memory (RAM) 134, and the input/output (I/O) circuit 128, all of which are interconnected via an address/data bus 136. It should be appreciated that although only one processor 132 is shown, the controller 110 may include multiple microprocessors 132. Similarly, the memory 130 of the controller 110 may include multiple RAMs 134 and multiple program memories 130. Although the I/O circuit 128 is shown as a single block, it should be appreciated that the I/O circuit 128 may include a number of different types of I/O circuits. The RAM(s) 134 and the program memories 130 may be implemented as semiconductor memories, magnetically readable memories, and/or optically readable memories, for example.

A link 138, which may include one or more wired and/or wireless (Bluetooth, WLAN, etc.) connections, may operatively connect the controller 110 to the fragrance dispersal system 108 through the I/O circuit 128. The fragrance dispersal system 108 may be in fluid communication with the environment 118 for purposes of distributing fragrance 107. A link 140, which may include one or more wired and/or wireless (Bluetooth, WLAN, etc.) connections, may operatively connect the controller 110 to the navigation system 104 through the I/O circuit 128. A link 142, which may include one or more wired and/or wireless (Bluetooth, WLAN, etc.) connections, may operatively connect the controller to the fragrance housing 106. In general, any sensor 112 is operatively connected to the controller 110 through the I/O circuit 128 by a link, such as link 138, link 140, or link 142. For example, a sensor 112 in the fragrance housing 106 is operatively connected to the controller 110 to communicate sensor data relating to, for example, the amount of fragrance 107 remaining in the fragrance housing 106. Other links, such as a link to battery 116, are contemplated as within the scope of this disclosure.

The program memory 130 and/or the RAM 134 may store various applications (i.e., machine readable instructions) for execution by the processor 132. For example, a user input system (such as user input system 202 discussed further below) may generally control the operation of the fragrance distribution drone 100 and the controller 110 to implement the processes described herein. The program memory 130 and/or the RAM 134 may also store a variety of subroutines 146 for accessing specific functions of the controller 110.

By way of example, and without limitation, the subroutines 146 may include, among other things:
a subroutine for receiving control commands from the user input system 150,
a subroutine for controlling the fragrance dispersal system 108 and the navigation system 104 to distribute a fragrance at a predetermined time while executing a flight pattern,
a subroutine for controlling the fragrance dispersal system 108 and the navigation system 104 to distribute a fragrance at a target location while executing a flight pattern,
a subroutine for controlling the fragrance dispersal system 108 and the navigation system 104 to achieve a target fragrance concentration at a target location while executing a flight pattern,
a subroutine for controlling the fragrance dispersal system 108 and the navigation system 104 to distribute a fragrance while executing a flight pattern in response to an activating event,
a subroutine for controlling the fragrance dispersal system 108 and the navigation system 104 to distribute a plurality of fragrances simultaneously at a target ratio while executing a flight pattern,
a subroutine for controlling the fragrance dispersal system 108 and the navigation system 104 to distribute a plurality of fragrances sequentially while executing a flight pattern,
a subroutine for adjusting operating parameters of the navigation system 104, such as propeller speed, quantity of propellers of a plurality of propellers in operation, and location of propellers of a plurality of propellers in operation, in response to the sensor 112 data indicating the change in the quantity of fragrance within the fragrance housing,
a subroutine for, in response to sensor data from sensor 112, controlling at least one of the fragrance dispersal system 108 and the navigation system 104 to distribute a fragrance,
a subroutine for, in response to sensor data from sensor 112, controlling at least one of the fragrance dispersal system 108 and the navigation system 104 to adjust a rate of distribution of a fragrance,
a subroutine for, in response to sensor data from sensor 112, controlling at least one of the fragrance dispersal system 108 and the navigation system 104 to cease distribution of a fragrance,
a subroutine for, in response to sensor data from sensor 112, controlling at least one of the fragrance dispersal system 108 and the navigation system 104 to adjust a rate of distribution or cease distribution of at least one of the plurality of fragrances set to achieve the target ratio,
a subroutine for, in response to sensor data from sensor 112, controlling at least one of the fragrance dispersal system 108 and the navigation system 104 to adjust the target ratio,
a subroutine for, in response to sensor data from sensor 112, controlling at least one of the fragrance dispersal system 108 and the navigation system 104 to adjust the predetermined time at which a fragrance is to be distributed while executing a flight pattern,
a subroutine for, in response to sensor data from sensor 112, controlling at least one of the fragrance dispersal system 108 and the navigation system 104 to adjust the target location,
a subroutine for, in response to sensor data from sensor 112, controlling at least one of the fragrance dispersal system 108 and the navigation system 104 to trigger a flight pattern of the navigation system,
a subroutine for, in response to sensor data from sensor 112, controlling at least one of the fragrance dispersal system 108 and the navigation system 104 to adjust the flight pattern of the navigation system,
a subroutine for, in response to sensor data from sensor 112, controlling at least one of the fragrance dispersal system 108 and the navigation system 104 to end the flight pattern of the navigation system, and/or
a subroutine to control the fragrance dispersal system 108 and the navigation system according to one of a plurality of modes, the plurality of modes including an intensive mode for rapid fragrance distribution and a maintenance mode for sustaining a consistent fragrance concentration,
a subroutine for flying the fragrance distribution drone within an environment based on a baseline flight plan,
a subroutine for collecting sensor data from sensor 112 during the baseline flight plan,
a subroutine for analyzing, using a processor 132 or 222, the sensor data,
a subroutine for optimizing, using a processor 132 or 222, a subsequent flight plan and operation of the fragrance dispersal system 108 based on the sensor data to achieve at a target fragrance concentration within the environment 118 while avoiding obstacles within the environment 118,
a subroutine for operating the fragrance distribution drone based on the optimized subsequent flight plan and the optimized operation of the fragrance dispersal system to achieve the target fragrance concentration within the environment while avoiding obstacles within the environment.

The program memory 130 and/or the RAM 134 may further store data related to the configuration and/or operation of the fragrance distribution drone 100, and/or related to the operation of the one or more subroutines 146.

In addition to the controller 110, the fragrance distribution drone 100 may include other hardware resources. As discussed in greater detail below, the fragrance distribution drone 100 may communicate with a user input system 150 through a network 152, using any of a number of known networking devices and techniques (e.g., through a computer network, the Internet, Bluetooth, NFC, short range wireless communication systems, etc.). A user input system 150 may be, for example, a mobile telephone, a computer, a tablet, a wearable device, a smart watch, a smart television, a gaming console, or an interactive kiosk. Specifically, the fragrance distribution drone 100 may include a drone wireless communication system 154 connected to the drone body 102 and in communication with the controller 110 via a link 127. The wireless communication system 154 may communicate with the user input system 150 via the network 152. Specifically, the drone controller 110 may receive control commands generated by and/or received by the input controller 150, or may generate control commands for the drone 100 based on data received from the input controller 150.

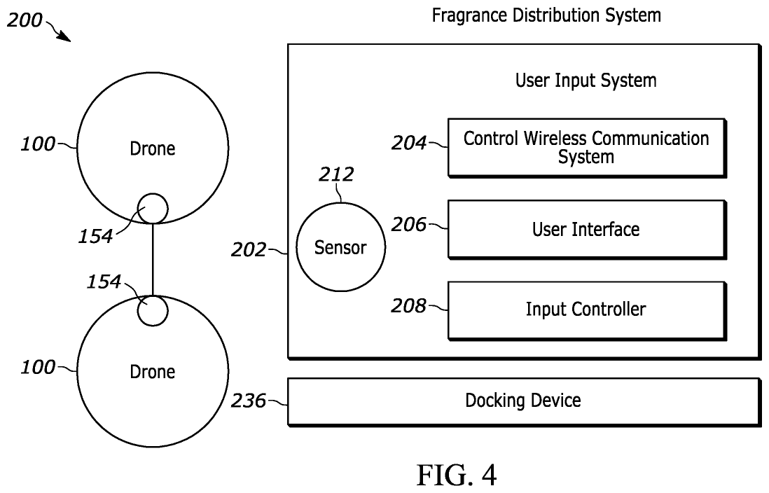

As shown in FIG. 4, the fragrance distribution drone 100 may be part of a fragrance distribution system 200. The fragrance distribution system 200 of FIG. 4 is shown with two fragrance distribution drones 100. The fragrance distribution system 200 may include only one fragrance distribution drone 100 or more than two fragrance distribution drones 100. As shown in FIG. 4, the fragrance distribution drones 100 may be in communication via their wireless communication systems 154. In addition to one or more fragrance distribution drones 100, the fragrance distribution system 200 includes a user input system 202 including a control wireless communication system 204 selectively in communication with the drone wireless communication system(s) 154, a user interface 206, and an input controller 208 in communication with the control wireless communication system 204. The fragrance distribution system 200 may further include a docking device 236 discussed further below.

An activating event may trigger the input controller 208 to activate the user interface 206. For example, an activating event may be user input at the user interface 206. Alternately, the fragrance distribution system 200 may include a sensor 212 for electronic recognition of a person or object by the user input system 202 or by a fragrance distribution drone 100 (using a sensor 112). In response to the activating event, or in response to a selection at the user interface 206, the drone controller 110 may be configured to execute any of the subroutines 146 discussed above.

The user interface 206 of the fragrance distribution system 200 is configured to receive user input including a selection of at least one of: a fragrance type, a target fragrance concentration, a target ratio of a plurality of fragrances, a sequential order for distribution of a plurality of fragrances, a predetermined time for fragrance distribution, a target location for fragrance distribution, and an activating event for fragrance distribution. These selections can then be implemented via subroutines 234 (discussed below) and subroutines 146 executed by the drone controller 110 and the input controller 208.

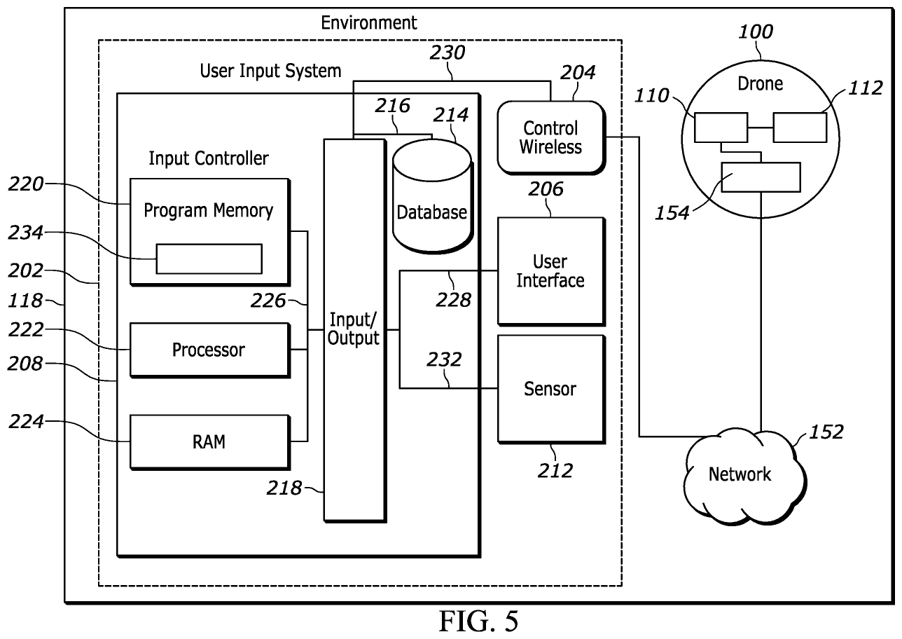

FIG. 5 illustrates schematically the input controller 208 of the user input system 202. The controller 208 is configured to execute the functions of the disclosed embodiments in order to control operation of the fragrance distribution drones 100. The input controller 208 may be operatively connected to a database 214 via a link 216 connected to an input/output (I/O) circuit 218. The database 214 may store, for example, sensor data. It should be noted that, while not shown, additional databases may be linked to the input controller 208 in a known manner. The input controller 208 includes a program memory 220, one or more processors 222 (may be called microcontrollers or a microprocessors), a random-access memory (RAM) 224, and the input/output (I/O) circuit 218, all of which are interconnected via an address/data bus 226. It should be appreciated that although only one processor 222 is shown, the input controller 208 may include multiple microprocessors 222. Similarly, the memory 220 of the input controller 208 may include multiple RAMs 224 and multiple program memories 220. Although the I/O circuit 218 is shown as a single block, it should be appreciated that the I/O circuit 218 may include a number of different types of I/O circuits. The RAM(s) 224 and the program memories 220 may be implemented as semiconductor memories, magnetically readable memories, and/or optically readable memories, for example.

A link 228, which may include one or more wired and/or wireless (Bluetooth, WLAN, etc.) connections, may operatively connect the input controller 208 to the user interface 206 through the I/O circuit 218. A link 230, which may include one or more wired and/or wireless (Bluetooth, WLAN, etc.) connections, operatively connects the input controller 208 to the control wireless communication system 204. The control wireless communication system 204 may communicate with the drone wireless communication system 154 via the network 152. Specifically, the control wireless communication system 204 may send control commands generated by and/or received by the input controller 208 to the drone 100 by communicating with the drone wireless communication system 154 via the network 152. Further, any sensor 212 is operatively connected to the input controller 208 through the I/O circuit 128 by a link 232. Other links, such as a link to a power source for the user input system 202, are contemplated as within the scope of this disclosure. Any sensor 112 of drone 100 may be in communication with the input controller 208 via the communication between the drone wireless communication system 154 and the control wireless communication system 204.

The program memory 220 and/or the RAM 224 may store various applications (i.e., machine readable instructions) for execution by the processor 222. The program memory 220 and/or the RAM 224 may also store a variety of subroutines 234 for accessing specific functions of the input controller 208. By way of example, and without limitation, the subroutines 234 may include, among other things:
a subroutine for receiving user selections or inputs,
a subroutine for generating control commands for the fragrance distribution drone 100 based on the user selections or input and/or sensor data,
a subroutine for storing a selection at the user interface to a user profile,
a subroutine for storing a plurality of user profiles,
a subroutine for, in response to drone information (e.g., battery level, fragrance quantity onboard the fragrance distribution drone 100, a maintenance reminder, fragrance usage patterns, and drone operation times) provided by the drone controller 110 (via the wireless communication system 154 and the control wireless communication system 204), displaying the drone information on the user interface 206 or sending the drone information via the control wireless communication system 204 (e.g., to a user's personal electronic device).

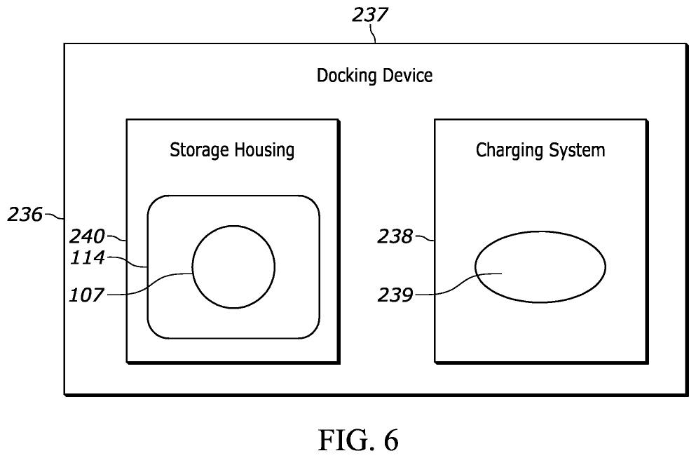

FIG. 6 illustrates schematically a docking device 236 configured to connect to the fragrance distribution drone 100. For example, the docking device may include a surface 237 on which a drone 100 may land. The docking device 236 includes a charging system 238 configured to charge the battery 116 of a drone 100. The charging system 238 may include, for example, inductive charging coils for inductively charging the battery 116 or direct electrical connections for wired charging of the battery 116. The charging system 238 is configured to connect to an outside power source, such as a battery or electrical connection, via an outlet 239. The docking device 236 may also include a storage housing 240 configured to store fragrance cartridge(s) 114 and/or liquid fragrance 107.

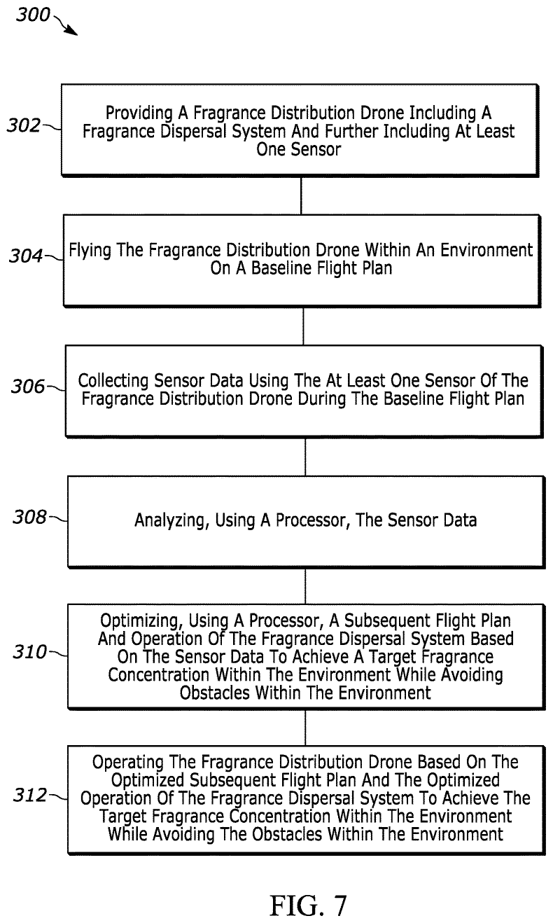

FIG. 7 illustrates schematically a drone-based method 300 of fragrance distribution. At box 302, the method 300 includes providing a fragrance distribution drone 100 including a fragrance dispersal system 108 and further including at least one sensor 112. At box 304, the method 300 includes flying the fragrance distribution drone 100 within an environment 118 on a baseline flight plan. At box 306, the method 300 includes collecting sensor data using the at least one sensor 112 of the fragrance distribution drone 100 during the baseline flight plan. For example, onboard LIDAR and radar sensors may be used to create a detailed 3D map. At box 308, the method 300 includes analyzing, using a processor 132 or 222, the sensor data. For example, machine learning and AI algorithms may be used to map the sensor data to paths that are designed to ensure even fragrance distribution. At box 310, the method 300 includes optimizing, using a processor 132 or 222, a subsequent flight plan and operation of the fragrance dispersal system 108 based on the sensor data to achieve at a target fragrance concentration within the environment 118 while avoiding obstacles within the environment 118. For example, the optimized operation may take into account real time sensor data relating to the current weight of the drone 100, the amount of fragrance 107 dispersed, and how this affects the drone's balance and flight dynamics. As another example, the optimization may consider the power required for stable flight and may redistribute power throughout the drone 100 to ensure efficient flight while compensating for changes in weight distribution. Using machine learning, the drone 100 may improve its predictive capabilities over time, learning from each flight to better anticipate and compensate for, for example, the effects of ballast changes on flight stability. This may involve learning the patterns of fragrance dispersing in different mission profiles and adjusting flight behavior preemptively during future missions. This also allows changes in viscosity and other characteristics of fragrances 107 without the need for physical modifications to drone 100. At box 312, the method includes operating the fragrance distribution drone based on the optimized subsequent flight plan and the optimized operation of the fragrance dispersal system to achieve the target fragrance concentration within the environment while avoiding obstacles within the environment.

The method 300 may further optionally include flying a plurality of fragrance distribution drones 100 simultaneously on coordinated baseline flight plans, collecting sensor 112 data using at least one sensor 112 of each of the plurality of fragrance distribution drones 100, and/or optimizing, using a processor 132 or 222, the subsequent flight plan of each of the plurality of fragrance distribution drones 100 based on the sensor data to achieve at a target fragrance concentration within the environment 118 while avoiding obstacles within the environment.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . aa”, “has . . . aa”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A fragrance distribution drone comprising a drone body,
a navigation system connected to the drone body;
a fragrance housing connected to the drone body;
a fragrance dispersal system connected to the drone body; and
a controller in communication with the navigation system and the fragrance dispersal system, the controller including a non-transitory memory and one or more processors;
wherein the non-transitory memory stores instructions that, when executed by the one or more processors, cause the controller to control the fragrance dispersal system and the navigation system to at least one of:
distribute a fragrance at a predetermined time while executing a flight pattern,
distribute a fragrance at a target location while executing a flight pattern,
achieve a target fragrance concentration at a target location while executing a flight pattern,
distribute a fragrance while executing a flight pattern in response to an activating event,
distribute a plurality of fragrances simultaneously at a target ratio while executing a flight pattern, and
distribute a plurality of fragrances sequentially while executing a flight pattern.

2. The fragrance distribution drone of claim 1 further comprising
at least one sensor configured to collect sensor data and provide the sensor data to the controller, the sensor data indicating a change in a quantity of fragrance within the fragrance housing; and
wherein the instructions, when executed by the one or more processors, further cause the controller to adjust operating parameters of the navigation system in response to the sensor data indicating the change in the quantity of fragrance within the fragrance housing.

3. The fragrance distribution drone of claim 2, the operating parameters including at least one of a propeller speed, quantity of propellers of a plurality of propellers in operation, and location of propellers of a plurality of propellers in operation.

4. The fragrance distribution drone of claim 1, further comprising
at least one sensor configured to collect sensor data and provide the sensor data to the controller, the sensor data comprising at least one of: a location of the drone, a type of detected fragrance, a fragrance concentration, a proximity to a person or object, an identification of a person or object, a rate of environmental airflow, and a direction of environmental airflow.

5. The fragrance distribution drone of claim 4, wherein in response to the sensor data, the instructions, when executed by the one or more processors, further cause the controller to control at least one of the fragrance dispersal system and the navigation system to at least one of:
distribute a fragrance,
adjust a rate of distribution of a fragrance,
cease distribution of a fragrance,
adjust a rate of distribution or cease distribution of at least one of the plurality of fragrances set to achieve the target ratio,
adjust the target ratio,
adjust the predetermined time at which a fragrance is to be distributed while executing a flight pattern,
adjust the target location,
trigger a flight pattern of the navigation system,
adjust the flight pattern of the navigation system, and
end the flight pattern of the navigation system.

6. The fragrance distribution drone of claim 1, the fragrance housing configured to selectively secure and release a fragrance cartridge.

7. The fragrance distribution drone of claim 1, wherein the instructions, when executed by the one or more processors, further cause the controller to control the fragrance dispersal system and the navigation system according to one of a plurality of modes, the plurality of modes including an intensive mode for rapid fragrance distribution and a maintenance mode for sustaining a consistent fragrance concentration.

8. A drone-based fragrance distribution system comprising:
a fragrance distribution drone comprising:
a drone body,
a navigation system connected to the drone body,
a fragrance housing connected to the drone body,
a fragrance dispersal system connected to the drone body,
a drone wireless communication system connected to the drone body, and
a drone controller in communication with the navigation system, the fragrance dispersal system, and the wireless communication system, the drone controller including a non-transitory memory and one or more processors; and
a user input system comprising:
a control wireless communication system selectively in communication with the drone wireless communication system,
a user interface; and
an input controller in communication with the control wireless communication system, the input controller including a non-transitory memory and one or more processors.

9. The drone-based fragrance distribution system of claim 8, the user interface configured to receive user input including a selection of at least one of: a fragrance type, a target fragrance concentration, a target ratio of a plurality of fragrances, a sequential order for distribution of a plurality of fragrances, a predetermined time for fragrance distribution, a target location for fragrance distribution, and an activating event for fragrance distribution.

10. The drone-based fragrance distribution system of claim 9, wherein the non-transitory memory of the drone controller or the non-transitory memory of the input controller stores instructions that, when executed by the one or more processors of the drone controller, cause the drone controller, in response to the selection at the user interface, to control at least one of the fragrance dispersal system and the navigation system to at least one of:
distribute a fragrance,
adjust a rate of distribution of a fragrance,
cease distribution of a fragrance,
adjust a rate of distribution or cease distribution of at least one of the plurality of fragrances set to achieve the target ratio,
adjust the target ratio,
adjust the predetermined time at which a fragrance is to be distributed while executing a flight pattern,
adjust the target location,
trigger a flight pattern of the navigation system,
adjust the flight pattern of the navigation system, and
end the flight pattern of the navigation system.

11. The drone-based fragrance distribution system of claim 9, wherein the input controller is configured to store a selection at the user interface to a user profile, and wherein the non-transitory memory of the input controller stores instructions that, when executed by the one or more processors of the input controller, cause the input controller to activate the user interface when triggered by an activating event.

12. The drone-based fragrance distribution system of claim 11, wherein the input controller is configured to store a plurality of user profiles.

13. The drone-based fragrance distribution system of claim 11, wherein an activating event is user input at the user interface or electronic recognition of a person or object by the user input system or by the fragrance distribution drone.

14. The drone-based fragrance distribution system of claim 12, wherein the non-transitory memory of the drone controller or the non-transitory memory of the input controller stores instructions that, when executed by the one or more processors of the drone controller, cause the drone controller, in response to the input controller activating the user interface, to control at least one of the fragrance dispersal system and the navigation system to at least one of:
distribute a fragrance,
adjust a rate of distribution of a fragrance,
cease distribution of a fragrance,
adjust a rate of distribution or cease distribution of at least one of the plurality of fragrances set to achieve the target ratio,
adjust the target ratio,
adjust the predetermined time at which a fragrance is to be distributed while executing a flight pattern,
adjust the target location,
trigger a flight pattern of the navigation system,
adjust the flight pattern of the navigation system, and
end the flight pattern of the navigation system.

15. The drone-based fragrance distribution system of claim 14, further comprising a docking device configured to connect to the fragrance distribution drone, a rechargeable battery connected to the drone body, and the docking device configured to electrically charge the rechargeable battery.

16. The drone-based fragrance distribution system of claim 14, wherein the fragrance dispersal system includes a plunger to increase pressure within the fragrance housing and a tube to siphon a fragrance from the fragrance housing.

17. The drone-based fragrance distribution system of claim 16, wherein the navigation system includes a propeller operably connected to a motor, and wherein the motor of the navigation system is also operably connected to the plunger of the fragrance dispersal system.

18. The drone-based fragrance distribution system of claim 8,
wherein the non-transitory memory of the drone controller stores instructions that, when executed by the one or more processors of the drone controller, cause the drone controller to provide drone information and send the drone information via the drone wireless communication system to the control wireless communication system, and
wherein the non-transitory memory of the input controller stores instructions that, when executed by the one or more processors of the input controller, cause the input controller to display the drone information on the user interface or send the drone information via the control wireless communication system.

19. The drone-based fragrance distribution system of claim 18, wherein the drone information is at least one of: a battery level, fragrance quantity onboard the fragrance distribution drone, a maintenance reminder, fragrance usage patterns, and drone operation times.

20. The drone-based fragrance distribution system of claim 18 comprising a plurality of fragrance distribution drones.

21. A drone-based method of fragrance distribution comprising:
providing a fragrance distribution drone including a fragrance dispersal system and further including at least one sensor;
flying the fragrance distribution drone within an environment on a baseline flight plan;
collecting sensor data using the at least one sensor of the fragrance distribution drone during the baseline flight plan;
analyzing, using a processor, the sensor data;
optimizing, using the processor, a subsequent flight plan and operation of the fragrance dispersal system based on the sensor data to achieve at a target fragrance concentration within the environment while avoiding obstacles within the environment; and
operating the fragrance distribution drone based on the optimized subsequent flight plan and the optimized operation of the fragrance dispersal system to achieve the target fragrance concentration within the environment while avoiding the obstacles within the environment.

22. The drone-based method of fragrance distribution of claim 21, further comprising
flying a plurality of fragrance distribution drones simultaneously on coordinated baseline flight plans;
collecting the sensor data using at least one sensor of each of the plurality of fragrance distribution drones; and
optimizing, using a processor, the subsequent flight plan of each of the plurality of fragrance distribution drones based on the sensor data to achieve at a target fragrance concentration within the environment while avoiding obstacles within the environment.
